(19) United States
(12) Patent Application Publication
(10) Pub. No.: US 2007/0010026 A1
(43) Pub. Date: Jan. 11, 2007

(54) Method of analysis of amine by mass spectrometry

(76) Inventors: Hoa Duc Nguyen, Orange, CA (US); Trinh Duc Nguyen, Anaheim, CA (US); Duc Tien Nguyen, Fountain Valley, CA (US)

Correspondence Address: Hoa Nguyen, Suite 225, 14441 Beach Blvd, Westminster, CA, 92683, US

(21) Appl. No.: 11/486,418

(22) Filed: Jul. 12, 2006

Related U.S. Application Data
(63) Continuation of application No. 10/619,709, filed on Jul. 14, 2003.

(51) Int. Cl. G01N 33/00

(52) U.S. Cl. 436/111

(57) ABSTRACT

Method of identification and quantitative analysis of primary and/or secondary amine(s) in a sample by mass spectrometry using stable isotope labeled internal standard is provided. Said internal standard is prepared by reaction of an authentic sample of said amine with a stable isotope labeled reagent, and is added to a sample containing said amine. Said amine in said sample is then quantitatively converted to a chemical compound of identical structure, except the stable isotope atoms, as that of said internal standard using a non-labeled reagent. Said sample is then extracted and the extract is analyzed by mass spectrometry. Identification and quantification of said amine are made from a plot of ion ratio of said converted amine to said internal standard versus amine concentration.

### CROSS-REFERENCE TO RELATED APPLICATIONS

Not applicable

### STATEMENT REGARDING FEDERALLY SPONSORED RESEARCH OR DEVELOPMENT

Not applicable

### BACKGROUND OF THE INVENTION

This invention pertains to methods of quantitative analysis of amines in a sample by isotope dilution mass spectrometry. The stable isotope labeled amides are used as internal standards. The sample may be a biological fluid, such as serum, urine etc., or an aqueous sample such as an environmental or an agricultural sample.

While various methods of analysis such as immunoassays and chromatographic analysis—LC (liquid chromatography), GC (gas chromatography), and TLC (thin layer chromatography)—have been reported for identification and determination of levels of amines in analytical samples, the absolute and unequivocal identification and quantitative analysis of those compounds are combinations of chromatographic analysis and MS (mass spectrometry) such as GC-MS and LC-MS. The accuracy and precision of these methods are usually the highest when stable isotope analogs of the analytes are used as internal standards. The mass spectrometry method of analysis using stable isotope internal standards is commonly called isotope dilution mass spectrometry. This method takes advantage of the similar chemical and physical behaviors of analytes and their respective isotope labeled internal standards towards all phases of sample preparation and also towards instrument responses. It uses the mass differentiation between analytes and their respective internal standard in mass spectrometry for quantification. The requirement for this method of analysis is the availability of stable isotope labeled internal standards.

The commonly used stable isotope labeled internal standard of an analyte is a chemical compound that has the same chemical structure as that of the analyte except that one or more substituent atoms are stable isotopes. Four commonly used stable isotopes are deuterium, carbon-13, nitrogen-15, and oxygen-18. For every hydrogen atom that is replaced by a deuterium atom, the molecular weight of resulting chemical compound is increased by one mass unit. This is also true for replacing a carbon atom with a carbon-3 atom, or by replacing a nitrogen atom with a nitrogen-15 atom. In the case of replacing an oxygen atom with an oxygen-18 atom, the molecular increase is two mass units. Although the acceptable stable isotope labeled internal standard for isotope dilution mass spectrometry method is the one that is not contaminated with any of the unlabeled material, the ideal one should be the one with the highest isotopic purity and contains as many stable isotope atoms as possible. The ideal one, however, must not contain any labeled isotope that can be exchanged for the unlabeled isotope under particular sample preparation conditions.

These criteria of an ideal stable isotope labeled internal standard present a challenge for organic synthesis chemists who help the analytical chemists in the analysis. Most often the synthesis of stable isotope internal standards is not simply an isotope exchange reaction. Easily exchangeable atoms are usually avoided due to possible re-exchange during sample preparation steps. Organic chemists often have to carry out multi-step synthesis to make stable isotope labeled internal standards. Even though many stable isotope labeled reagents are commercially available, the choice of appropriate labeled reagent for chemical synthesis of stable isotope labeled internal standards is still very limited. The limited isotope labeled reagents and the multi-step synthesis contribute to the high cost of synthesis of stable isotope internal standards. Even if the analytical chemist who carries out the analysis can afford the cost of the synthesis, there is also a time factor that he or she has to consider before ordering the synthesis. Situations where organic chemists spent weeks and months on a synthesis project and came up with nothing at the end were common. This invention offers a solution for this problem.

The objective is a short and reliable method of preparing a stable isotope labeled internal standard that is suitable for the analysis of an analyte in question, but not the synthesis of the stable isotope labeled analyte. Within the context of the isotope dilution mass spectrometry method, both analyte and its internal standard have to have identical chemical structures, with the exception of the isotope atoms which provide the mass differentiation upon mass spectrometric analysis. Analytical chemists who uses GC-MS for their analysis often “derivatize” the analyte and its stable isotope labeled analyte (used as internal standard) into chemical compounds that can easily pass through the GC column or else provide better instrumental responses. The analysis becomes the analysis of the “derivatized” analyte and the “derivatized” internal standard, but still provides comparably accurate results of concentrations of the analyte itself. Examples of these analyses are found in cited references. Using similar reasoning, one can synthesize a stable isotope derivative of the analyte by reacting it with a stable isotope labeled reagent. The resulting isotope labeled chemical compound can be used as internal standard in the analysis of the analyte, providing that the analyte in the analyzed sample will be converted to a chemical compound of identical structure as that of the internal standard using a non-labeled reagent. There are 3 requirements for the usefulness of this method:
1. The analyte in the sample must be quantitatively converted to the compound of identical structure (except the labeled atoms) as that of the added isotope labeled internal standard using a non-labeled reagent.
2. Absolutely no conversion of the isotope labeled internal standard to the non-labeled compound because the conversion of the analyte happens in the sample in the presence of the added isotope labeled internal standard.
3. The conversion of the analyte into the compound of identical structure as that of the added isotope labeled internal standard has to be accomplished before any isolation method i.e. extraction, is performed.

The first two requirements relate to the chemistry of the analyte in question. The efficiency of a chosen chemical reaction depends on the type of reaction which, in turn, depends on the type of functional groups of the analyte. This invented method relates to the analysis of primary and secondary amines whose chemistry focuses on the reactivity of the primary and secondary amino functional groups of the analyte.

Quantitative reactions of primary and secondary amines in aqueous samples include conversion reactions to an amide using an acid anhydride or an acid chloride.

There are other reactions of primary and secondary amines that are very efficient, but the above conversion reactions are very efficient in aqueous environment and can be performed at room temperature and in a relatively short reaction time. These are necessary and practical features for routine analysis of primary and secondary amines in aqueous samples.

### BRIEF SUMMARY OF THE INVENTION

The current invention provides for a method of identification and quantification of primary amine(s) or secondary amine(s) in a sample by isotope dilution mass spectrometry. The stable isotope labeled internal standard(s) of said amine(s) is synthesized beforehand by reacting a sample containing the analyzed amine(s) with a labeled reagent. Following this step, said stable isotope labeled internal standard(s) is then added to a sample containing the analyzed amine(s). The analyzed amine(s) is then converted to a non labeled analog(s) of said labeled internal standard(s) with identical chemical structure as said labeled internal standard(s) except for the stable isotope atoms using a non-labeled reagent. Both converted analyzed amine(s) and its corresponding said stable isotope labeled internal standard(s) are then extracted and analyzed by mass spectrometry. The stable isotope labeled internal standard(s) provided in the current invention are labeled amide(s). The type of labeled internal standard(s) used will dictate the labeled reagents used for its synthesis as well as the non-labeled reagent used to convert the analyzed amine(s) to the corresponding analog(s).

In comparison with the traditional method of isotope dilution mass spectrometric analysis of more than one amines, the invented method offers the following advantages:
1. The efficiency and simplicity of the above reactions makes possible the short, reliable, and quick synthesis of individual stable isotope labeled internal standards, whereas in the traditional method of analysis, stable isotope labeled internal standard of each amine has to be independently synthesized.
2. It is possible to quickly and efficiently synthesize a library of stable isotope internal standards for the analysis of an entire library of amines using these reactions and only one commercially available stable isotope labeled reagent.
3. Because the synthesis of stable isotope labeled internal standard in this invented method is usually a one-step synthesis, the entire process of synthesis and sample preparation can be performed in an automated fashion. The internal standard is prepared in one step, excess isotope reagent is then destroyed, and the prepared internal standard can be added directly to the samples without purification. The non-labeled reagent is added and the sample is ready for extraction shortly thereafter.
   These attractive features make the method suitable for high throughput analysis of amines by isotope dilution mass spectrometry.

### DETAILED DESCRIPTION OF THE INVENTION

The current invention provides for a method of identification and quantification of primary amine(s) or secondary amine(s) in a sample by mass spectrometry. Said primary amine(s) or secondary amine(s) has the following formulas R₁NH₂ and R₁R₂NH, wherein R₁ and R₂ are alkyl, aryl, and heteroatom containing cyclic or non-cyclic groups. The current method comprises, as an integral part of the analysis of said amines, the following steps:
1. Synthesizing labeled amide internal standard(s) by reacting an authentic sample of said primary or secondary amine(s) with a stable isotope labeled reagent to form said amide internal standard(s) of the general formulas R₁NHCOR₃ or R₁R₂NCOR₃, wherein R₃ is a stable isotope labeled alkyl or aryl group. Said R₃ stable isotope labeled alkyl or aryl group is selected from the group consisting of CD₃, CD₂CD₃ or C₆D₅. Said stable isotope labeled reagent is a labeled acid anhydride or an acid chloride selected from the group consisting of labeled acetic acid anhydride, labeled propionic acid anhydride and labeled benzoic acid anhydride or labeled acetyl chloride, labeled propionyl chloride, and labeled benzoyl chloride.
2. A known amount of said stable isotope labeled amide internal standard(s) was then added to said sample containing said amine(s) to be analyzed.
3. Said sample was then contacted with a non-labeled acid anhydride or an acid chloride selected from said group consisting of acetic acid anhydride, propionic acid anhydride and benzoic acid anhydride or acetyl chloride, propionyl chloride, and benzoyl chloride to quantitatively convert said primary or secondary amine(s) in the sample into said amide(s) of identical structure as that of said amide internal standard(s) mentioned above except for the stable isotope atoms.
4. Appropriate extraction methods were then used to isolate said amide(s) and their corresponding amide internal standard from said sample. Concentration of said amine(s) were determined and quantified by mass spectrometry and based on the ratio of said converted amide(s) and their corresponding amide internal standard.

### EXAMPLE

### Analysis of Paroxetin in Human Serum Plasma

Step 1: Preparation of N-Acetylparoxetin-d3.

A solution of 10 mg of paroxetin in ethyl acetate was treated with 2 equivalents of acetic anhydride-d6. The resulting solution was stirred for an hour then was quenched with aqueous sodium carbonate. The aqueous phase was extracted with ethyl acetate and the combined organic phases were dried with magnesium sulfate. The filtered solution was concentrated and the residue was purified by column chromatography using silica gel as absorbant and methanol-chloroform mixture as eluant. The fractions containing clean N-acetylparoxetin-d3 were combined and concentrated to give 4 mg product as a white solid. MS analysis gave MH+375.

Step 2: Preparation of Working Standard Solutions and Internal Standard Solution.

Working standard solutions of Paroxetin were prepared by weighing paroxetin and diluting the stock solution to appropriate concentration as follows:

| | | | |
|---|---|---|---|
| Solution | A | 0.1 | ug/ml in ethyl acetate |
| | B | 0.2 | ug/ml |
| | C | 0.5 | ug/ml |
| | D | 2.0 | ug/ml |
| | E | 5.0 | ug/ml |
| | F | 15.0 | ug/ml |
| | G | 20.0 | ug/ml |

Working quality control standard solutions of Paroxetin were prepared by independently weighing paroxetin and diluting the stock solution to appropriate concentration as follows:

| | | | |
|---|---|---|---|
| QC Solution | J | 0.3 | ug/ml in ethyl acetate |
| | K | 6.0 | ug/ml |
| | L | 14.0 | ug/ml |

Working internal standard solution of Paroxetin were prepared by weighing N-acetylparoxetin-d3 and diluting the stock solution to a working concentration of 10 ug/ml in ethyl acetate.

Step 3: Preparation of Calibration Samples and Quality Control Samples in Human Plasma.

Paroxetin-free human plasma aliquots of 0.1 ml were treated with 100 ul of solution A to G to make calibration samples A to G.

Paroxetin-free human plasma aliquots of 0.1 ml were treated with 100 ul of solution J to L to make quality control samples J to L.

Both calibration samples and quality control samples were then treated with 100 ul of the internal standard working solution.

A human plasma aliquot of 0.1 ml was treated with 100 ul of the internal standard solution to make the “zero” sample.

Another human plasma aliquot of 0.1 ml was not treated with 100 ul of the internal standard solution to make the “blank” sample.
Step 4: Sample Treatment and Extraction.

To all prepared samples were added 100 ul of a 10% v/v acetic anhydride in ethyl acetate. The samples were mixed and left standing at room temperature for 15 minutes. Aqueous sodium carbonate and sodium bicarbonate 0.5 ml were added to each sample to quench excess acetic anhydride. The samples were extracted with 0.5 ml ethyl acetate. Each extract was separated and concentrated. The residue of each extract was reconstituted with 100 ul of acetonitrile.
Step 5: Analysis of Reconstituted Extracts by LC/MS/MS.

A total of 12 reconstituted extracts were loaded on a Perkin Elmer autosampler that was connected to a Perkin Elmer LC pump and a PE Sciex API 365 MS. Each extract was run through an Inersil column of Sum at a rate of 0.5 ml/min of acetonitrile/water 50/50 mixture. The eluate was directly fed to the MS ion source. MS data were collected for 1.5 min per injection.

MS analysis was performed in MRM mode. m/z 372.2>m/z 192.0 was monitored for N-acetylparoxetin while m/z 375.2>m/z 193.0 was monitored for N-acetylparoxetin-d3. Collected data were ploted against concentration using McQuan 1.5 sofware.

Results are tabulated as follows:

Paroxetin

Internal Standard: is

Weighted (1/x*x)

Intercept=−6.015

Slope=0.552

Correlation Coeff.=0.998

Use Area

| | | | | Calc. | |
|---|---|---|---|---|---|
| Filename | Filetype | Accuracy | Conc. | Conc. | Ratio |
| ParoAc A | Standard | 102.171 | 0.100 | 0.102 | 0.042 |
| ParoAc B | Standard | 96.445 | 0.200 | 0.193 | 0.092 |
| ParoAc blank | Blank | n/a | 0.0 | n/a | n/a |
| ParoAc C | Standard | 100.094 | 0.500 | 0.500 | 0.261 |
| ParoAc D | Standard | 91.128 | 2.000 | 1.823 | 0.991 |
| ParoAc E | Standard | 98.434 | 5.000 | 4.922 | 2.702 |
| ParoAc F | Standard | 102.226 | 15.000 | 15.334 | 8.449 |
| ParoAc G | Standard | 109.502 | 20.000 | 21.900 | 12.073 |
| ParoAc QC J | QC | 90.106 | 0.300 | 0.270 | 0.134 |
| ParoAc QC K | QC | 100.755 | 6.000 | 6.045 | 3.322 |
| ParoAc QC L | QC | 103.010 | 14.000 | 14.421 | 7.945 |
| ParoAc zero | Standard | n/a | 0.0 | n/a | n/a |

We claim:

1. A method of identification and quantification of amine in a sample comprising the steps of:
a) combining a known amount of an amide internal standard with said sample comprising said amine;
b) contacting said sample with an acid anhydride or an acid chloride to convert said amine in said sample into an amide of identical structure as that of said amide internal standard except for the stable isotope atoms;
c) extracting said sample to isolate said amide and said amide internal standard; and
d) analyzing said amide and said amide internal standard by mass spectrometry.

2. The method of claim 1 wherein the concentration of said amine in said sample is determined and quantified by isotope dilution mass spectrometry using isotope labeled internal standard.

3. The method of claim 1 wherein said amine is a primary amine or a secondary amine having the following formula R₁NH₂ and R₁R₂NH wherein R₁ and R₂ are alkyl, aryl, and heteroatom containing cyclic or non-cyclic groups.

4. The method of claim 1 wherein said amide internal standard is a stable isotope labeled internal standard.

5. The method of claim 1 wherein said amide internal standard is synthesized by reacting an authentic sample of said amine with a stable isotope labeled reagent to form said amide internal standard having the following formula R₁NHCOR₃ or R₁R₂NCOR₃, wherein R₃ is a stable isotope labeled alkyl or aryl group.

6. The method of claim 1 wherein the extraction step c) can be any appropriate separating methods such as solid phase extraction, liquid-liquid extraction or solid supported liquid-liquid extraction.

7. The method of claim 1 wherein said acid anhydride is selected from a group consisting of acetic acid anhydride, propionic acid anhydride, and benzoic acid anhydride.

8. The method of claim 1 wherein said acid chloride is selected from a group consisting of acetyl chloride, propionyl chloride, and benzoyl chloride.

9. The method of claim 1 wherein said sample contains either a singularity or a plurality of primary amines and/or secondary amines.

10. The method of claim 1 wherein there is no conversion of said stable isotope labeled amide internal standard to its corresponding non-labeled amide compound during step b).

11. The method of claim 1 wherein the converting step b) is performed in an aqueous environment.

12. The method of claim 1 wherein the converting step b) is performed before the extraction step.

13. The method of claim 1 wherein the converting step b) is quantitative.

14. The method of claim 5 wherein said stable isotope labeled alkyl group and aryl group are selected from a group consisting of CD3,CD2CD3 and C6D5 respectively.
